(19) United States
(12) Patent Application Publication
(10) Pub. No.: US 2023/0390509 A1
(43) Pub. Date: Dec. 7, 2023

(54) METHOD OF OPERATING A VENTILATOR

(71) Applicant: Loewenstein Medical Technology S.A., Luxembourg (LU)

(72) Inventors: Jan SCHATTNER, Pinneberg (DE); Jan VERHOEVEN, Ettlingen (DE); Christof GOEBEL, Hamburg (DE)

(21) Appl. No.: 18/203,141

(22) Filed: May 30, 2023

(30) Foreign Application Priority Data
Jun. 1, 2022 (DE) 102022113877.1

(51) Int. Cl. A61M 16/00

(52) U.S. Cl. CPC A61M 16/0009; A61M 16/024; A61M 2016/0027; A61M 2016/003; A61M 2205/502

(57) ABSTRACT

System having at least one therapy device for performing a therapy and at least one hose system for the gas-conducting connection between the therapy device and a patient. The therapy device includes a source for gas for generating an overpressure and/or a drain for gas for generating a negative pressure to at least temporarily provide an overpressure and/or negative pressure to the hose system. The system furthermore comprises a control unit, a user interface, a memory, and a sensor, and the control unit includes at least one of the mechanisms therapy start mechanism, therapy reminder mechanism, therapy repetition mechanism, each of the mechanisms being based on user actions, patient data, and/or therapy data.

### CROSS-REFERENCE TO RELATED APPLICATIONS

The present application claims priority under 35 U.S.C. § 119 of German Patent Application No. 102022113877.1, filed Jun. 1, 2022, the entire disclosure of which is expressly incorporated by reference herein.

### BACKGROUND OF THE INVENTION

### 1. Field of the Invention

The invention relates to a system and a therapy device having mechanisms for automatically starting a therapy and/or secretion management or the automatic reminder thereof.

### 2. Discussion of Background Information

Mechanical insufflators/exsufflators are known in the prior art in order to assist patients in coughing up secretion. The mechanical insufflators/exsufflators are generally used independently of ventilators. For the therapy, it is necessary to change from the ventilator to the therapy device for this purpose. Since the mechanical insufflators/exsufflators are rarely used and the use is sometimes not a particularly pleasant experience, the patient can quickly forget a regular use. In this case, greater quantities of secretion can accumulate in the airways of the patient, which can result in worsening of the respiration situation. A prophylactic use of a mechanical insufflator/exsufflator or a prophylactic (manual) extraction of secretion improves the respiration situation here and has the result that the lungs are regularly freed of secretion. In addition to the insufflators/exsufflators, extraction devices are also known, which are used depending on the available time, for example, in the hospital to remove secretion from the upper airways or the mouth area, for example.

In view of the foregoing, it would be advantageous to have available a system for a regular and need-compliant secretion management.

### SUMMARY OF THE INVENTION

The present invention provides a system having at least one therapy device for performing the therapy, and at least one hose system for the gas-conducting connection between the therapy device and a patient, wherein the therapy device includes at least one source for gas for generating an overpressure and/or at least one drain for gas for generating a negative pressure and at least sometimes providing the hose system with an overpressure and/or negative pressure for performing the therapy, and wherein the system furthermore comprises at least one control unit, at least one user interface, at least one memory, and at least one sensor. The system is characterized in that the control unit includes a therapy start mechanism for starting the therapy and/or a therapy reminder mechanism for reminding of the therapy and/or a therapy repetition mechanism for extending and/or repeating the therapy, which are each based on user actions, patient data, and/or therapy data.

In some embodiments, the system is characterized in that the control unit is configured to make the reminder of the therapy and/or the start of the therapy and/or the repetition and/or the extension of the therapy adjustable via the user interface and/or an external interface.

In some embodiments, the system is characterized in that the therapy start mechanism and/or the reminder mechanism and/or the therapy repetition mechanism can be canceled and/or shifted in time via the user interface.

In some embodiments, the system is characterized in that the control unit is configured to automatically adapt for a frequency of the reminders and/or start of the therapy via the user interface and/or via the interface and/or data characterizing the patient from the memory, for example for the lung function (vital capacity/insufflation capacity, lung/thorax complaints, etc.), age, weight, respiration data, data on the pathology/underlying illness, prior experience with coughing maneuvers.

In some embodiments, the system is characterized in that, on the basis of determined data, in particular based on measured values and stored values for SpO2, pulse, and/or CO2 and/or changing pressure and volume flow behavior, the therapy is automatically started or the start of the therapy is offered and/or reminded to the patient if these data indicate that a secretion removal from the airways by a therapy would be required.

In some embodiments, the system is characterized in that the therapy device is coupled to a ventilator or the therapy device and the ventilator are combined as one device and the data are provided by the ventilator.

If the therapy device only generates overpressure, it is reasonable if the therapy device and the ventilator are embodied as the same device. For example, the therapy device can be embodied as at least one hardware and/or software module of the ventilator.

In some embodiments, the system is characterized in that the underlying data in particular comprise the SpO2 value, wherein upon reaching a threshold value of the SpO2 saturation, a therapy start and/or a therapy reminder and/or a therapy repetition and/or a therapy extension takes place.

In some embodiments, the system is characterized in that the SpO2 threshold value is stored in the system and is optionally adjustable.

In some embodiments, the system is characterized in that the effect of the therapy performed on the course of at least one of the variables SpO2, CO2, pressure, flow, tidal volume, acoustic signals/tones, signal-to-noise ratio (SNR) is stored and/or processed in the therapy device and/or in a coupled ventilator and wherein these stored and/or processed data are taken into consideration in decisions on the therapy start and/or therapy repetitions and/or therapy reminders.

In some embodiments, the system is characterized in that the system is configured to evaluate the underlying data, in particular the signal-to-noise ratio (SNR) of the pressure and/or flow signals, and, upon reaching a threshold of the SNR value, initiates or extends the therapy start or a reminder thereof.

In some embodiments, the system is characterized in that the reminder or the option for starting the maneuver exclusively and/or additionally takes place via the coupled ventilator.

In some embodiments, the system is characterized in that the therapy start and/or the therapy reminder and/or the therapy repetition change certain parameters on the ventilator.

In some embodiments, the system is characterized in that a determined effect of therapies performed on the course of at least one of the variables SpO2, CO2, flow, tidal volume, signal-to-noise ratio (SNR) change the ventilation parameters on the ventilator.

In some embodiments, the system is characterized in that the therapy device and the ventilator can be pneumatically coupled, so that ventilation and machine insufflation and exsufflation are directly combined.

In some embodiments, the system is characterized in that the two device functions ventilation and machine insufflation/exsufflation are implemented in one device.

In some embodiments, the system is characterized in that, on the basis of the determined data, in particular based on measured values and stored values for pressure, flow, tidal volume, SpO2, pulse, and/or CO2 or data from prior cough maneuvers, such as the peak cough flow (PCF) or the point in time of the therapy starts and/or reminders and/or therapy repetitions, repetitions of insufflation and exsufflation are automatically performed and/or offered to the patient when these data indicate that further secretion is to be removed from the airways.

In some embodiments, the system is characterized in that determined and/or stored data are uploaded into a cloud-based system and that cloud-based algorithms make therapy-related decisions and that these decisions are in turn provided to the therapy device from the cloud for the adaptation of the therapy or of starting times of the therapy.

In some embodiments, the system is characterized in that user-side feedback of the subjective perception of automatically performed therapy starts and/or repetitions and/or reminders are possible at the user interface and/or via an app on a mobile terminal coupled to the therapy device and wherein this user-side feedback is stored and/or processed and is taken into consideration in following decisions on the therapy start and/or therapy repetitions and/or reminders of a therapy start.

In some embodiments, the system is characterized in that the cough therapy can be started and/or repeated and/or ended on the user side as a result of a therapy reminder by the therapy device via a remote control and/or an app on a mobile terminal coupled to the therapy device.

In some embodiments, the system is characterized in that an automatic therapy start is provided on the basis of determined data, wherein the therapy parameters are automatically adapted on the basis of the determined data.

In some embodiments, the system is characterized in that a message, which indicates the automatic therapy start, is generated on the therapy device and/or a coupled ventilator.

In some embodiments, the system is characterized in that the automatic therapy start can be canceled and/or shifted in time via the user interface.

In some embodiments, the system is characterized in that the therapy device comprises at least one source for generating an overpressure, in order to at least temporarily provide overpressure for an insufflation to the hose system to perform the therapy.

In some embodiments, the system is characterized in that the therapy device comprises at least one drain for gas for generating a negative pressure, in order to at least temporarily provide negative pressure for extracting secretion and/or for an exsufflation to the hose system to perform the therapy.

In some embodiments, the system is characterized in that the therapy device comprises at least one source for gas for generating an overpressure and at least one drain for gas for generating a negative pressure and a switching means, wherein the switching means is configured to at least temporarily provide overpressure for an insufflation and/or negative pressure for an exsufflation to the hose system, wherein the control unit is configured at the hose system, to provide overpressure below a predetermined pressure and/or pressure profile and for a predetermined duration for the insufflation, and preferably then to provide negative pressure below a predetermined pressure and/or pressure profile and for a predetermined duration for the exsufflation.

In some embodiments, the system is characterized in that at least one control unit, at least one memory, at least one user interface, and at least one sensor are part of the therapy device.

The invention furthermore relates to a therapy device, comprising at least one source for gas for generating an overpressure, wherein the therapy device furthermore comprises at least one control unit, a user interface, a memory, and a sensor, wherein the therapy device is connectable to at least one hose system for connecting a patient to the therapy device and the therapy device includes at least one switching means, which is configured to provide an overpressure for insufflation at least temporarily at the hose system. The therapy device is characterized in that the control unit includes a therapy start mechanism for starting the therapy and/or a reminder mechanism for reminding of the therapy and/or a therapy repetition mechanism for extending and/or repeating the therapy, which are each based on user actions, patient data, and/or therapy data.

In some embodiments, it is characterized in that the therapy device furthermore comprises a drain for gas for generating a negative pressure, wherein the switching means is configured to provide overpressure for an insufflation and/or negative pressure for an exsufflation at least temporarily to the hose system and the control unit is configured,
a. at the hose system, to provide overpressure below a predetermined pressure and/or pressure profile and for a predetermined duration for the insufflation, and
b. preferably then to provide negative pressure below a predetermined pressure and/or pressure profile and for a predetermined duration for the exsufflation.

In some embodiments, it is characterized in that the therapy device also comprises a function for ventilation and/or can be coupled to a ventilator.

It is to be noted that the features listed individually in the claims can be combined with one another in any technically reasonable manner and disclose further embodiments of the invention. The description in particular additionally characterizes and specifies the invention in conjunction with the figures.

It is furthermore to be noted that a conjunction “and/or” used herein, which is between two features and links them to one another, is always to be interpreted so that in a first embodiment of the subject matter according to the invention only the first feature can be present, in a second embodiment only the second feature can be present, and in a third embodiment both the first and the second feature can be present.

A ventilator is to be understood as any device which assists a user or patient in natural breathing, takes over the respiratory therapy of the user or living being (e.g. patient and/or newborn baby and/or premature baby) and/or is used for respiratory therapy and/or influences the breathing of the user or patient in another way. This includes, for example, but not exclusively, CPAP and BiLevel devices, narcosis or anesthesia devices, respiratory therapy devices, (clinical, nonclinical, or emergency) ventilators, high flow therapy devices. Ventilators can also be understood as diagnostic devices for respiratory therapy. Diagnostic devices can be used in general for acquiring medical and/or breathing-related parameters of a living being. These also include devices which can acquire and optionally process medical parameters of patients in combination with or exclusively relating to the breathing.

A patient interface, if not expressly described otherwise, can be understood as any peripheral device which is intended for interaction, in particular for therapy or diagnostic purposes, of the measuring device with a living being. In particular, a patient interface can be understood as a mask of a ventilator or a mask connected to the ventilator. This mask can be a full face mask, thus enclosing nose and mouth, or a nose mask, thus a mask only enclosing the nose. Tracheal tubes and cannulas and so-called nasal cannulas can also be used as a mask or patient interface. In some cases the patient interface can also be a simple mouthpiece, for example a tube, through which the living being at least exhales and/or inhales.

In the meaning of the invention, a source for gas for generating an overpressure is synonymous with a source for overpressure gas. A drain for gas for generating a negative pressure is synonymous with a source for negative pressure gas. It is to be expressly noted that a source for overpressure gas can also simultaneously represent a source for negative pressure gas. Depending on the embodiment, a common source for overpressure gas and negative pressure gas is thus to be found. Alternatively or additionally, however, separate sources can also be arranged for overpressure gas and negative pressure gas.

In the course of the description of the invention, the term “therapy”, if not described otherwise, generally designates secretion management. The secretion management can comprise, for example, a cough therapy, a cough assistance, an insufflation, an insufflation with following exsufflation, and/or also the extraction of secretion from the mouth or the airways. The extraction of secretion via a cannula, for example in patients who are ventilated via a tracheal cannula, can also be viewed as a therapy in the meaning of the invention. The treated patient is, for example, moved and/or assisted to cough for this purpose here by an insufflation, optionally with subsequent exsufflation. The coughing can be used in this case, for example, to cough up or remove secretion from the airways and/or the lungs. A therapy generally comprises multiple cough maneuvers here, which are in turn divided into multiple cough cycles. A cough cycle generally comprises at least one insufflation and one expiration or coughing here. In some embodiments, a cough cycle comprises at least one insufflation and one exsufflation.

An insufflation describes a pressure-assisted deepened inspiration of the patient, wherein the inspiration pressure and possibly the inspiration length are greater than in the case of a normal inspiration in the context of a (machine) ventilation. In relation thereto, flows and/or volumes can also be greater. The insufflation can also be performed over multiple steps, for example, between which a pause (air stacking) lies instead of the expiration. The insufflation can alternatively be performed in a single stroke. The single stroke can be extended and/or deepened in relation to the normal inspiration, for example. After the insufflation, for example, an exsufflation follows or the patient can perform a deep and forced expiration (coughing).

An exsufflation describes a provision, optionally sudden, of negative pressure (in relation to the normal ambient pressure) after the insufflation, by which a deep expiration or a cough of the patient is forced. The provision of a negative pressure results in a higher pressure difference here and can enable more efficient coughing.

The therapy start can be characterized by various aspects, which are partially dependent on the respective conditions. If the therapy device is an independent device, so that patient interface and/or hose have to be changed, a therapy start can be viewed, for example, as the change of the patient interface. A therapy start can also be defined by the switching on and/or preparation of the therapy device. In some embodiments, the therapy start is the point in time from which the therapy device starts and/or prepares the first cough maneuver. A preparation can be, for example, one or more breaths before the first cough cycle. The warming up and/or starting up of the therapy device and/or activation of the fan can also be viewed as the therapy start.

The therapy end can be defined by ending the last therapy cycle. It can also be viewed as the therapy end when the therapy device is switched off or deactivated. If the therapy device is, for example, connected to a ventilator at least for data transfer, a deactivation of the therapy device is thus the state when the therapy device no longer actively carries out a therapy and/or only still exchanges data with the coupled ventilator. A change or removal of the patient interface for the cough therapy can also be considered the therapy end.

The system comprises at least one mechanism for reminding of a therapy and/or a mechanism for starting a therapy and/or a mechanism for repeating and/or extending a therapy. It can be provided that the system comprises two or all three of the mechanisms. The mechanisms can be optionally activated and/or deactivated here. It can also be provided that at least one of the mechanisms is automatically activated and/or deactivated. If a therapy device is coupled with a ventilator, for example, at least one of the mechanisms can be activated. In this case, an activation/deactivation does not mean that a therapy is performed immediately, but rather that the mechanism now activated can decide when a therapy is to be performed and possibly starts it or generates a corresponding notification.

The system comprises in this case at least one control unit, at least one memory, at least one user interface, and at least one sensor. The control unit is configured and designed here so that it includes the therapy start mechanism and/or the therapy repetition mechanism and/or the therapy reminder mechanism. In some embodiments, the control unit is part of the therapy device. However, it can also be provided that the control unit is arranged independently at least in location from the therapy device in the system. For example, the control unit can be arranged in a ventilator which is optionally coupled with the therapy device.

The system comprises at least one therapy device which is designed for performing the therapy.

In some embodiments, the system comprises at least one therapy device which is designed to generate an overpressure by means of at least one source for gas and to apply this overpressure to a hose system. The therapy device is configured, for example, to perform an insufflation using the overpressure. The system additionally comprises, for example, a switching means, which stops the application of overpressure to the hose system by a sudden changeover, due to which a sudden pressure drop results in patients and possibly brings the patient to a deep forced expiration or a cough.

In some embodiments, the therapy device alternatively or additionally comprises a drain for gas for generating a negative pressure. Secretion can be extracted from the airways of a patient via the negative pressure in some embodiments. The drain can also be combined in some embodiments with a source for gas for generating an overpressure, so that the drain can also be used simultaneously or temporarily as a source for gas. For example, a therapy device can simultaneously or temporarily also be used for ventilation, for example with overpressure, of a patient, and can be switched over for the secretion management. In some embodiments, the negative pressure can be provided to the hose system for an exsufflation.

In some embodiments, the system comprises at least one therapy device, which is designed to generate an insufflation and/or an exsufflation by means of at least one controllable source or drain for negative pressure and/or overpressure. In some embodiments, the therapy device comprises a source for gas for generating an overpressure and a drain for gas for generating a negative pressure. It can be provided here that the source and drain are combined, the source can thus also be used simultaneously as the drain. The therapy device is thus designed to assist a patient in coughing and/or to induce a cough of the patient. The goal of coughing is, for example, secretion management, among other things coughing up or removing secretion from the airways (including lungs) of the patient. It can also be provided that the therapy device is designed to perform the coughing for the patient. It can be controlled via a switching means whether overpressure gas or negative pressure gas is supplied to the patient.

Mechanism for Reminding of the Therapy

In some embodiments, the system comprises a mechanism for reminding of the therapy, the therapy reminder mechanism. The reminder mechanism is designed so that a reminder of the therapy is performed on the basis of a data situation. Such a reminder can be designed in various forms, which are optionally combinable with one another.

In one form of the reminder, a message and/or an alarm is generated by the system, for example, which notifies the patient and/or caregiver that a therapy should and/or must take place. Such a message can be output, for example, via a user interface. It can be provided here that the message solely contains a notification that a therapy is to be performed. In some embodiments, the message/the alarm contains additional items of information. These additional items of information can be, for example, a recommended form of therapy and/or one or more reasons for the therapy and/or notifications on the next steps for performing the therapy. Reasons for performing are, for example, a duration since the last time a therapy was performed and/or a medical relevance, such as secretion accumulation in the airways and/or a regular secretion management. In addition, medical relevance can be provided if an illness, such as an infection, was present or is present. Furthermore, other medical measurements such as respiratory flow, SpO2, CO2, blood pressure, blood sugar level, or taking medications could influence the reminder.

The notifications on the next steps of the therapy can contain, for example, instructions for performing it. These instructions are optionally gone through step-by-step together with the actions of the patient and/or caregiver. If the current instruction step provides, for example, that a patient interface is to be connected, the instructions thus pass to the next step when it is detected that a patient interface has been connected. For actions which the system cannot independently detect, it can be provided that a confirmation via a user interface is provided.

It can alternatively or additionally be provided that the notability of the reminder is graduated. If the reminder is based, for example, on a duration since the last therapy, at the earliest provided point in time for the therapy, a simple reminder can be generated which contains a notification, for example, that a certain duration since the last therapy has passed and/or that a therapy can or should be performed. This first simple reminder can be canceled, ignored, and/or postponed by the patient and/or user, for example. After further duration, for example, a more notable reminder can be generated, so that the patient and/or caregiver can recognize that performing the therapy is becoming more acute, thus the therapy should be performed soon or promptly.

It can be provided here that a minimum and maximum duration between two therapies is provided. For example, it can be provided that the closer the current duration is to the maximum duration, the more notable the reminders become. The reminders can become more notable, for example, due to a color design, font size, animations (for example flashing of the display), and/or acoustic assistance. If, for example, the maximum duration since the last therapy is reached, the reminder can be designed in the form of an alarm, which is significantly more perceptible, for example due to an acoustic alarm.

In some embodiments, it can be provided that the reminders can be postponed. If a reminder is generated by the mechanism, but the patient and/or the caregiver is not ready for a therapy, for example, the reminder can be acknowledged by a confirmation of the reminder, for example, so that a reminder is generated again at a later point in time. It can also be provided that upon reaching a maximum duration since the last therapy, the reminder cannot be further postponed. If a special escalation stage is provided—for example by acoustic signals—in some embodiments the reminder can merely be switched to silent, but not deactivated.

A reminder of the therapy is provided at least on the therapy device. However, the reminder can also be expanded to further devices. For example, a coupling of the therapy device with a ventilator can be provided, in which data are exchanged at least temporarily between the devices. It can be provided here that the reminder is additionally or alternatively generated on the ventilator, the patient and/or the caregiver is thus reminded of performing a therapy via the ventilator. A reminder can also be generated and/or displayed via external remote stations, such as an app on a mobile terminal and/or a computer, for example connected to a cloud.

In some embodiments, the patient and/or caregiver is prompted to select settings for the therapy following the reminder and/or together with the reminder. It can be provided here that at least the start of the therapy can be selected. Alternatively or additionally, the patient and/or user is prompted to select the therapy form and/or to input and/or select various parameters for the therapy.

Mechanism for Starting the Therapy

In some embodiments, the system alternatively or additionally comprises a mechanism for starting therapy, the therapy start mechanism. The starting mechanism is designed so that a start of the therapy takes place on the basis of a data situation. Such a start can be designed in various forms, which are optionally combinable with one another.

One form of the therapy start is preparation of the therapy. If the system detects, for example, on the basis of the data situation that a therapy start is to take place, a start can take place in the meaning of a preparation of the therapy. The preparation comprises, for example, loading a therapy program, which contains, for example, a number of maneuvers and/or cycles. Other parameters for the therapy, such as the intensity of the therapy, can also be stored in a therapy program. Alternatively or additionally, the preparation of the therapy comprises making the therapy device ready, for example by starting up and/or warming up. One embodiment of the preparation can also be distinguished in that only a start option is provided for the patient and/or caregiver. For example, a start button can be released and/or displayed on a touch sensitive display screen (touchscreen). In some embodiments, it can also be provided that the system is prepared by the therapy start mechanism and performing the therapy is started via a remote station, for example an app on a mobile terminal.

In addition, it can also be provided that the preparation of the therapy displays a display of steps to be performed for the therapy. These steps can be, for example, changing a patient interface and/or inputting or querying additional specifications/data/information/parameters for the therapy.

The starting mechanism can alternatively or additionally also provide that the system is put into a state in which the patient and/or caregiver only has to actuate the start button so that the therapy is performed.

In some embodiments, a therapy device is coupled with a ventilator in the system so that at least a data exchange takes place. It can be provided by way of example that in times in which the therapy device is not used, a display of the user interface of the therapy device is used to expand a display of the ventilator. The therapy start mechanism can have various effects on this coupling in this case.

For example, it can be provided that the start mechanism for the start of the therapy ends at least an expanded display. Alternatively or additionally, it can be provided that an additional display with respect to the therapy start is generated on the display of the ventilator. If the expanded display of the ventilator is ended by the start mechanism, it can be provided that displays corresponding to the therapy start are displayed on the display of the therapy device.

With a coupling of therapy device with ventilator in a form in which at least data are exchanged, it can also be provided that data of the ventilator can also be incorporated in the data situation which is used as the decision basis for the therapy start mechanism.

In some embodiments of the system, the therapy device is alternatively or additionally pneumatically coupled with a ventilator. A pneumatic coupling can provide here that only the patient interface has to be changed for the performance. It can also be provided that a patient interface is used which is suitable both for ventilation and also for a cough therapy. It can thus be provided that the therapy start mechanism automatically starts performing the therapy.

In a pneumatic coupling, for example, a valve can be provided, which switches between the pneumatics of the ventilator and the pneumatics of the therapy device. If the therapy start mechanism decides on the basis of the data situation that a therapy is to be performed, it can be provided, for example, that the therapy is automatically started. In this case, for example, a switch is made via the valve from the pneumatics of the ventilator to the pneumatics of the therapy device, so that the therapy can be performed. The therapy start can thus be started without action of the patient and/or a caregiver.

Alternatively or additionally, it can be provided that a message is generated via the therapy device and/or the ventilator which notifies the caregiver and/or the patient of the imminent therapy start. It can be provided, for example, that the patient and/or caregiver is made aware by visual notifications. It can also be provided that the patient and/or caregiver is notified by an audio signal of the upcoming therapy start. Optionally, multiple notifications can also take place, for example at specific time intervals before beginning the therapy. It can be provided, for example, that at a specific time interval to the therapy start, the therapy is announced via visual notifications. As the therapy start approaches, the visual notifications can be assisted by audio signals. The patient and/or caregiver is thus not surprised by a beginning therapy. The automatic start of the therapy can also comprise an automatic selection of a therapy program.

If it is established on the basis of the data situation by the system that an acute situation prevails, in which an immediate therapy start is advisable, it can also be provided that the therapy is started without further notifications. In some embodiments, it can be provided that in addition an alarm message is also generated which notifies of the acute situation, for example via a remote station such as a connected server and/or a mobile terminal.

In some embodiments, the system comprises a combined ventilator and therapy device. The mechanism for starting the therapy can act in this case as with a pneumatic coupling of a therapy device and a ventilator. A switch is made between a ventilation mode and a therapy mode in the combined device here to start the therapy.

Alternatively or additionally, a combination of the therapy start mechanism with a reminder mechanism can also be provided. Depending on the data situation, for example, a reminder of the therapy can first be initiated before the start of the therapy takes place. For example, a reminder of the therapy takes place at least once. If the user ignores the reminder and/or a point in time and/or a data situation is reached such that a beginning of the therapy is necessary, it can be provided that the change is made from the reminder mechanism to the start mechanism. The start mechanism can generate, for example, a notification that a therapy start will take place after a time span. After passage of this time span, the therapy starts in accordance with the described therapy start mechanism. With pneumatically coupled ventilator and therapy device and combined therapy device/ventilator, for example, performing the therapy can begin automatically. In contrast, with devices which are not coupled or are only coupled for a data exchange, it can be provided that the therapy start relates to the preparation of the therapy, e.g., warming up/starting up the device/individual components.

Mechanism for Repeating the Therapy

In some embodiments, the system alternatively or additionally comprises a mechanism for repeating the therapy, the therapy repetition mechanism. The therapy repetition mechanism decides on the basis of a data situation whether the therapy will be repeated and/or extended. The data situation can, for example, comprise data which are registered during a therapy presently being performed. It can be provided that to decide on the therapy repetition and/or therapy extension, it is assessed whether and/or to what extent the therapy achieves an effect. It can be detected, for example, that the patient displays too few or no coughs and/or other data indicate inadequate therapy. Inadequate therapy exists, for example, if the peak cough flow (PCF) was too low and/or certain pressures were not reached. In some embodiments, this can alternatively or additionally indicate an (excessively high) leakage or an excessively low oxygen saturation (SpO2). As a result, it can be provided that additional cough maneuvers and/or cough cycles are added to the present therapy in the form of a therapy extension. For example, cycles and/or maneuvers are added until a specific threshold value of coughs of the patient and/or other data is reached, which indicates at least an adequate therapy. Optionally, it can also be provided that the additional maneuvers and/or cycles have adjusted parameters. If the registered data during the present therapy indicate, for example, that the intensity of the cycles and/or maneuvers is too low to generate a cough, for example, in order to trigger a reaction of the patient by the change between overpressure and negative pressure, it can be provided that the following and/or added cycles and/or maneuvers have a correspondingly adjusted intensity.

Alternatively or additionally, a repetition of the therapy can also be provided. For example, the same therapy is performed again thereafter, optionally with an adjustable, fixed, and/or automatically determined time delay. In some embodiments, it can also be provided that instead of the same therapy, an at least partially changed therapy is performed as a repetition. For example, the changes can be based at least partially on data which are registered during the original therapy.

In some embodiments, a therapy combination mechanism can be provided, which combines the functions of at least two of the above-mentioned mechanisms. For example, the functions of the reminder mechanism and the start mechanism are combined in the combination mechanism. A combination of reminder and repetition mechanism and/or a combination of repetition and start mechanism are also conceivable. In some embodiments, the therapy combination mechanism comprises the functions of the therapy reminder, therapy start, and therapy repetition mechanisms.

Coupling of Therapy Device and Ventilator

In some embodiments, the system also comprises, in addition to a therapy device, a ventilator which is coupled with the therapy device. A coupling for the data exchange between the devices and/or a pneumatic coupling of the devices can be provided.

With a coupling of the devices for data exchange, the therapy device and the ventilator are connected to one another via a data line. This data line can be a wired connection and/or a wireless connection. Due to the data exchange, both devices can access the data which are registered by the respective other device. For example, a ventilator is used over a greater period of time than the therapy device and can thus also register a more extensive set of data. In particular, the ventilator registers data during the ventilation of a patient. These data can also be evaluated, for example, as to whether a therapy using the therapy device is reasonable and/or should be performed. In some embodiments, the therapy device is configured such that in the case of a coupling with a ventilator, the display of the ventilator can be at least temporarily expanded via the display of the user interface of the therapy device.

With a coupling for data exchange, it can also be provided that some calculations and/or evaluations of data of the ventilator are performed on the therapy device. For example, an evaluation of the data to judge whether a therapy is necessary can be performed on the therapy device. In some embodiments, the ventilator can be configured so that it independently carries out an evaluation to judge the necessity of a therapy and passes on a corresponding result to the therapy device. The data of the ventilator can alternatively or additionally also be used by the therapy device to adapt parameters of the therapy and/or to select a therapy form or a therapy program.

In some embodiments, it is provided that data which are registered by the therapy device during the therapy are at least partially evaluated as to whether and/or how the ventilation using the ventilator can be adapted. The evaluation can take place for this purpose on the therapy device and/or the ventilator. The data registered by the ventilator can thus influence the therapy and/or the data registered by the therapy device can influence the ventilation.

In some embodiments of the system, each therapy device is alternatively or additionally pneumatically coupled with a ventilator. The pneumatic coupling can take place independently of or jointly with a coupling for data exchange. In a pneumatic coupling, it can be provided, for example, that the hose system between patient and device does not have to be exchanged, but rather a switch is made between the pneumatics of the ventilator and the pneumatics of the therapy device. For example, a valve device is connected to both pneumatics and can be controlled by a control unit to switch between ventilation and therapy. Alternatively, a combined therapy device and ventilator can also be provided. A combined device comprises, for example, separate devices for performing the therapy and for ventilation and/or a device which can provide both the therapy and a ventilation.

Data Situation as the Basis

A data situation is used as the basis for the mechanism for therapy reminder and/or for therapy start and/or for therapy repetition. The data situation can comprise multiple data and parameters.

In some embodiments, the data situation, which is used as the basis for at least one of the mechanisms (therapy reminder, therapy start, therapy repetition), comprises at least one duration. The duration can be, for example, a duration since the last therapy. How the duration is assessed can, for example, be permanently specified and/or adjustable by the patient and/or caregiver and/or defined on the basis of the last and/or following therapy.

An (optionally repeating) therapy can be, for example, a regular secretion management, which is performed regularly to remove accumulating secretion from the airways of a patient. In a healthy person, this typically occurs almost unnoticed and/or without aid, for example, by deep inhalation and/or independent coughing. In some people, in contrast, it is necessary to assist this secretion management or perform it mandatorily. The secretion management can comprise here, for example, an insufflation with following coughing, a combined insufflation/exsufflation, and/or the extraction of secretion from the airways. A duration can be provided between the therapies, after which the therapy is performed again. The respective mechanism, for example the therapy reminder mechanism and/or the therapy start mechanism, can then, for example, react to a passed duration and activate the reminder and/or the therapy start. Alternatively or additionally, a time plan can also be provided or adjustable, which optionally provides a differing duration in each case between the therapies. Such a time plan can provide, for example, that during a waking phase of the patient, a shorter duration between the therapies is provided and during the sleeping phase, a longer duration is provided or the duration is selected so that no therapy is to take place during the sleeping phase.

The data situation can alternatively or additionally also comprise a therapy plan, in which points in time are defined at which a therapy reminder and/or a therapy start is triggered. It can also be provided that various therapy forms are stored in the therapy plan, which are provided at specific points in time. The defined point in time can in some embodiments also be a period of time within which the therapy is to be performed. For example, it can be provided for the therapy reminder mechanism that a first reminder is generated at the beginning of the period of time or at the point in time. With a combined embodiment of the reminder mechanism and the start mechanism, it can be provided that at the defined point in time, first a reminder is generated and after a defined and/or adjustable number of reminders, a therapy start is triggered.

In some embodiments, the data situation alternatively or additionally comprises data and/or information which are input by a patient and/or caregiver, for example via a user interface. These data and/or information can be, for example, patient data such as sex, age, height, weight, and/or illnesses. In some embodiments, it can be provided that the therapy reminder mechanism and/or the therapy start mechanism and/or the therapy repetition mechanism determines and/or recommends a duration which is between two therapies on the basis of the input data and/or information. It can also be provided that the therapy form is determined by the respective mechanism and/or a recommendation for the therapy form is generated. Furthermore, it can be provided that a patient and/or caregiver can input additional information and/or data between two therapies, which are taken into consideration for the next durations. For example, an infection in the patient can be determined and this can be input as information between two therapies, so that, for example, the duration between the therapies and/or the therapy form or therapy intensity is adapted and/or a recommendation for the adaptation is generated.

If it is input, for example, that the patient suffers from an infection, it can be provided that the duration between two therapies is shortened. In relation thereto, it can optionally be provided that in addition further adjustment options with respect to the therapy are offered. In some embodiments, various types and/or severities of infections can be specified, whereupon the respective mechanism performs and/or offers different changes of the duration and/or other therapy parameters.

In some embodiments, the data situation additionally or alternatively also comprises data registered by the therapy device. The data registered by the therapy device can be, for example, ventilation data and/or therapy data. During the therapy, for example, data, such as sensor data, can be registered and evaluated by the therapy device. These include, for example, at least flow and/or pressure and/or a frequency, for example breathing and/or coughing frequency. It can moreover be provided that the therapy device is designed to evaluate the registered data. On the basis of the evaluation of the data, the reminder mechanism and/or the start mechanism and/or the repetition mechanism can decide when and/or whether a therapy is provided and/or repeated and/or extended. For example, it can be provided that a live evaluation of the data takes place and the therapy repetition mechanism can decide on the basis of the evaluation whether a repetition or extension of the therapy is necessary. It can also be provided that the therapy repetition mechanism alternatively or additionally outputs a recommendation for repetition and/or extension, which can be accepted and/or modified by the patient and/or caregiver. It can also be provided that the therapy reminder mechanism and/or the therapy start mechanism can adapt the duration until the next therapy on the basis of the data registered during the therapy, optionally in combination with further data and information. It can also be provided that the therapy form is adapted on the basis of the registered data.

If it is detected during the evaluation of the data, for example by an evaluation unit and/or one of the mechanisms, that an inadequate number and/or intensity of coughs takes place, it can be provided that the therapy repetition mechanism provides an extension and/or repetition of the therapy. It can also be provided here that the repetition mechanism does not provide an immediate repetition of the therapy, rather a time delayed repetition. Alternatively or additionally, it can be provided that the repetition mechanism defines or adjusts a shortening of the duration until the next therapy. For the therapy reminder mechanism and/or the therapy start mechanism, it can be provided that in the case of inadequate coughs, the duration until the next therapy is shortened and/or the therapy form or therapy intensity is adapted.

For example, it can be provided that the cough flow is detected, in particular the peak values of the cough flow (PCF, peak cough flow). The PCF can provide indications, for example, as to whether a cough maneuver and/or a cough cycle was effective. For example, the therapy repetition mechanism can decide on the basis of the PCF whether the coughing was too weak and the therapy will be repeated and/or extended. In the case of an extension, for example, further cough maneuvers and/or cycles are appended to the current therapy.

In some embodiments, it can alternatively or additionally be provided that the data situation comprises data registered by the ventilator. These can include, for example, sensor data and values which are registered by the ventilator during the ventilation. The consideration of data of the ventilator can take place in particular in the case of a coupling for data transmission between therapy device and ventilator. On the basis of the data registered by the ventilator, it can be evaluated, for example, whether a therapy is necessary. For example, the ventilation data such as flow and/or pressure can indicate that a certain amount of secretion has collected in the airways of the patient and should be removed by a therapy. In some embodiments, the ventilation data can also indicate increasing worsening and/or improvement of the accumulation of secretion in the patient. In such cases, for example, it can be provided that the duration until the next therapy reminder and/or therapy start is automatically and/or dynamically adapted. It can also be provided here that the therapy form and/or therapy intensity is adapted.

It can be provided, for example, that an SpO2 value and/or a CO2 value and/or other blood gas values and/or other respiratory gas values are registered by the ventilator. It can be established on the basis of an evaluation of these values, for example, that coughing is necessary. One exemplary procedure is the method according to John R. Bach. In this case, it is taken into consideration that a drop of the SpO2 value below a threshold value in specific patient types, for example having prior illness, indicates an accumulation of secretion, thus coughing up is reasonable and/or necessary. The threshold value is, for example, between 90% and 98%, preferably between 93.5% and 96%. For example, the threshold value is 95% or 94%.

Additionally or alternatively, a signal-to-noise ratio (SNR), for example of the pressure and/or flow signal, can be registered and/or evaluated. It can be provided, for example, that a larger quantity of secretion is concluded in the case of a high SNR. This in turn has the result that the time interval until the next reminder and/or performance is shortened.

Additionally or alternatively, variations in the pressure and/or flow can be registered and/or evaluated. The registered variations can result, inter alia, for example, due to flow separations and/or closures/re-openings and indicate a larger quantity of secretion. This evaluation can result, for example, in a shortening of the time interval until the next reminder and/or performance.

In some embodiments, acoustic signals and/or tones can alternatively or additionally also be registered and evaluated. It can thus be detected, for example, whether secretion or other liquids have collected in the lungs of the patient. For example, the breathing noises of the patient can be registered and evaluated.

In some embodiments, multiple data sources are used. It can be provided, for example, that a therapy urgency index is determined. The urgency index can be calculated, for example, from a duration since the last therapy and further data. A therapy reminder and/or a therapy start is then triggered, for example, when the urgency index reaches a defined, optionally adjustable threshold value. For example, it can be provided that the urgency index rises with increasing duration since the last therapy. Depending on the evaluation of further data, the urgency index can be additionally increased. If the evaluation of data has the result, for example, that secretion is increasingly accumulating in the airways of the patient, an additional increase of the urgency index can thus take place. Optionally, it can also be provided that a base value of the urgency index is determined on the basis of the data situation. If an infection of the patient exists, for example, it can be provided that the base value of the urgency index is higher than without infection. As a result, the threshold value is reached after a shorter duration.

In some embodiments, it is provided that the therapy device is configured to be able to perform a long-term prognosis and/or long-term evaluation. A higher level of accuracy of the need for therapy of a patient can be achieved via such a prognosis and/or evaluation, for example. For example, for this purpose it is evaluated how various therapy forms, therapy parameters, and/or therapy intensities affect the development of the patient. It can be provided, for example, that it is established by the prognosis and/or evaluation that certain therapy parameters have a positive influence on the patient, for example so that a therapy is required more rarely.

In some embodiments, it is alternatively or additionally provided that data registered by the therapy device and/or ventilator are uploaded into a cloud-based system. The cloud-based system is configured, for example, to make therapy-related decisions via algorithms, for example with regard to the frequency, form, and/or intensity of the therapy. These decisions are in turn loaded from the cloud onto the therapy device and/or ventilator, wherein an adaptation of the therapy parameters (e.g., frequency, form, intensity) is performed on the basis of the decisions on the therapy device and/or the ventilator. The therapy-related decisions can accordingly also cause adaptations of the therapy start, therapy reminder, and/or therapy repetition mechanism. For example, the starting point in time of the therapy and/or the duration between two therapies can be adapted.

According to the invention, a therapy reminder and/or a therapy start and/or a therapy repetition is performed on the basis of a data situation. Various data can be used here. For example, a therapy reminder can primarily be based on a duration, for example since the last therapy, and a therapy repetition on data during the current and/or last therapy, optionally supplemented with input data, such as patient parameters. A therapy start can also be triggered, for example, on the basis of ventilation data, inter alia, if these indicate that coughing up appears reasonable and/or necessary. However, a therapy start can also be triggered exclusively on the basis of a duration.

In some embodiments, it is alternatively or additionally provided that at least the therapy reminder mechanism comprises a type of waking function, wherein a reminder is output via the therapy device and/or and optionally coupled ventilator and/or a remote station on the basis of a data situation. Multiple reminder stages can optionally be provided, optionally also having escalating notifications in the form of image and/or sound. In some embodiments, the reminder can also give the patient and/or caregiver a selection option in order to select, for example, therapy form and/or therapy intensity and/or a time until the next reminder.

In some embodiments, it can alternatively or additionally be provided that the reminders of the therapy reminder mechanism become less notable over a longer period of time (multiple days/weeks/months). The goal can be, for example, that the patient becomes accustomed to a repeating therapy and independently begins to start the therapy regularly. It can optionally be provided that the notability of the reminders is reduced and/or is increased again if the patient and/or the caregiver forgets the therapy once and/or multiple times.

The coupling of therapy device and ventilator provides various options. A data exchange alone can take place, each device interprets the data for itself alone. With a data exchange alone, it can also be provided that reminders, etc. are displayed on the ventilator. In addition to a “wired coupling”, a coupling via radio/Bluetooth/WLAN can also be provided. A control of the therapy device via the ventilator can also be provided, for example a start/stop of the therapy. Mutual influencing is additionally also conceivable, therapy results/findings can result in a change of the ventilation and vice versa the therapy can be adapted on the basis of ventilation data (related to patient and device). Alternatively or additionally, it can also be provided that during the ventilation by the ventilator, thus in the time in which the therapy device is not used by the patient, the computing power of the therapy device can be used to perform evaluations and/or analyses, etc., of the ventilator.

A therapy start can follow the therapy reminder. After therapy start, a therapy repetition is possible. For example, after a duration, reminders take place until a threshold value of duration is reached. Therapy start is then decided. The “forced” therapy start can have various types, which are more reasonable with coupled devices. For example, with pneumatically coupled devices, after some time—after announcement—the therapy can simply be started without the patient explicitly requesting this. The therapy parameters can be adjusted here depending on the data situation, wherein it is also distinguished whether a forced start is present or the patient starts the therapy themselves.

### BRIEF DESCRIPTION OF THE DRAWINGS

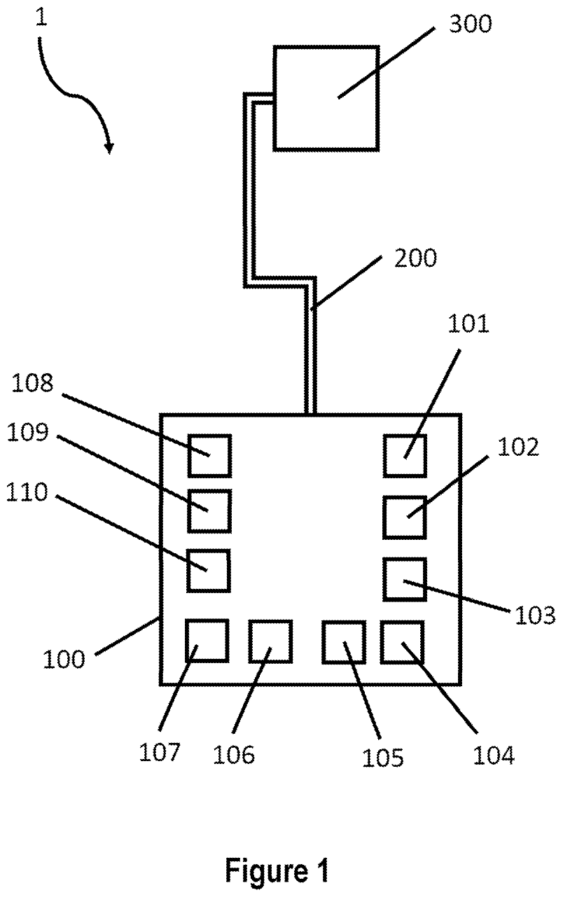

The invention is described in more detail by way of example on the basis of FIGS. 1 to 5.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

FIG. 1 shows a system 1 having a therapy device 100, which is connected via a hose system 200 to a patient 300.

The therapy device 100 comprises at least one controllable source for gas for generating an overpressure. The system 1 furthermore comprises switching means 102, via which overpressure can be applied to the hose system 200, for example to perform an insufflation. The system 1 having the switching means 102 is configured to suddenly stop the application of overpressure to the hose system 200 and to force an expiration and/or a cough of the patient 300.

In some embodiments, the therapy device 100 comprises at least one controllable pressurized gas source for generating an overpressure gas and a pressurized gas source for generating a negative pressure gas or at least one controllable source for gas for generating an overpressure and a controllable drain for gas for generating a negative pressure. For example, the therapy device 100 comprises a combined pressurized gas source 101 for generating a negative pressure gas and an overpressure gas or the one pressurized gas source 101 is used as a source and drain for gas for controllably generating an overpressure and/or negative pressure. For example, the pressurized gas source 101 comprises for this purpose at least one fan and a pneumatic system having switching means 102, which enables the fan to provide negative pressure gas or overpressure gas to the hose system 200 depending on the switching. Alternatively or additionally, two fans having opposing delivery direction can also be provided, which are connected in parallel, wherein it is controlled by switching means 102 whether negative pressure gas or overpressure gas is provided to the hose system 200.

The exemplary therapy device 100 furthermore comprises a memory 103 for storing data, values, and information, which can be input, for example, via a user interface 107, a sensor 105, an evaluation unit 106, and/or via a ventilator (not shown) coupled for data exchange. The memory 103 is designed to provide data, values and/or information to the control unit 104, the evaluation unit 106, the therapy reminder mechanism 108, the therapy repetition mechanism 109, and/or the therapy start mechanism 110. The therapy reminder mechanism 108, the therapy repetition mechanism 109, and/or the therapy start mechanism 110 can be provided, for example, as software modules on the evaluation unit 106 and/or the control unit 104. The therapy mechanisms 108, 109, 110 can in some embodiments also comprise separate hardware modules and/or can be installed on a shared hardware module. It can also be provided that data, values, and/or information can be displayed from the memory 103 via the user interface 107. The data, values, and information stored in the memory 103 can contain, inter alia, at least parameters on the therapy, such as therapy length, therapy intensity, and/or therapy form. Data with respect to the patient 300 can also be comprised.

For example, the therapy device 100 comprises three therapy mechanisms: a therapy reminder mechanism 108, a therapy repetition mechanism 109, and a therapy start mechanism 110. The individual mechanisms were already described above in detail and will only be explained briefly and by way of example at this point.

The therapy reminder mechanism 108 is configured to remind the patient 300 and/or a caregiver of the therapy on the basis of a data situation. For example, at least one duration since the last performed therapy is used as a data situation. The therapy reminder mechanism 108 generates for this purpose, for example, a corresponding notification, for example for display on the user interface 107. The notification can indicate, for example, that a specific duration since the last therapy has passed and accordingly a new therapy is to be started. The notification can also comprise step-by-step instructions for this purpose, which display the steps of the therapy, for example which preparations have to be made, etc. In some embodiments, it can be provided that the patient 300 or a caregiver can mark the reminder as seen and/or can postpone it, so that a new notification is generated after a specific duration.

In some embodiments, the therapy reminder mechanism 108 can also be configured to prepare the therapy device 100 for the therapy. For example, a therapy program can be loaded, therapy settings or therapy parameters can be automatically adjusted and/or the therapy device can be warmed up/started up. It can also be provided that the therapy reminder mechanism 108 prepares the therapy enough that the patient 300 and/or caregiver only has to confirm the performance of the therapy, for example via the user interface 107. As already explained above, in addition to the duration, other factors, data, values, and/or information can also be incorporated in the data situation.

The therapy repetition mechanism 109 is configured, for example, to start a repetition and/or an extension of the therapy on the basis of a data situation. The data situation can comprise in particular values of the sensor 105 here, which are registered during the therapy. For example, the therapy repetition mechanism 109 can conclude by an evaluation of the data that the therapy is not adequate or is not yet adequate. If the therapy is not adequate, a repetition can be provided. For the repetition, for example, the same therapy settings or parameters can be reused. However, it can also be provided that certain parameters, such as intensity and/or therapy form, are changed to achieve a better therapy. If the therapy is not yet adequate, it can be provided, for example, that the therapy is extended by at least one cycle, it can also be provided that it is extended by at least one or more maneuvers. In some embodiments, a protective mechanism is provided to prevent an excessively long therapy, even if it is not yet adequate. In this case, for example, a notification or an alarm can additionally be generated that a maximum therapy length is reached, but it is not yet adequate.

The therapy start mechanism 110 is configured, for example, to automatically start the therapy. The therapy start mechanism 110 can be constructed similarly to the therapy reminder mechanism 108 with respect to the data situation. An automatic start of the therapy is possible in particular if the therapy device 100 is pneumatically coupled with a ventilator 400 (not shown). The therapy start mechanism 110 can then provide an automatic start, for example, when a maximum duration and/or maximum number of reminders of the therapy have passed.

To perform the therapy, the pressurized gas source 101 and the switching means 102 are activated in accordance with the specifications, which are stored, for example, in the memory 103. During the performance of the therapy, first an overpressure gas for insufflation is provided to the patient 300 via the hose system 200, so that the patient 300 can inhale and air can pass behind the secretion to be coughed up. The provision of the overpressure gas is then stopped and/or a switch is made to the negative pressure gas suddenly by the therapy device 100 or by the switching means 102, so that a cough is triggered in the patient 300, by which the secretion is to be transported away. In the course of the therapy, multiple coughs or cycles are generally provided, wherein a cycle consists of an inhalation (inspiration) and an exhalation (expiration) or cough. Depending on the therapy settings, the intensity, such as the pressure difference and/or the switching speed, of the coughs can vary. A pause between 0.1 seconds and 10 seconds is generally provided between two therapy cycles. In some embodiments, a therapy can also be divided into multiple maneuvers, wherein a maneuver in turn comprises multiple cycles. It can also be provided that the pause is greater between two maneuvers them between two cycles, for example also greater than 10 seconds.

After ending the therapy, it can be provided that the patient 300 and/or a caregiver can input feedback on the therapy via the user interface 107, for example the subjective feeling of the effect of the therapy. A further performance of the therapy can also be requested.

In some embodiments, the system 1 or the therapy device 100 is designed to perform a ventilation of the patient. For example, an overpressure ventilation or a respiration assistance of the patient can be performed via the source for gas for generating an overpressure. In some embodiments, the therapy device 100 is thus a ventilator and therapy device combined. The control unit 104 is configured and designed here so that it activates the further modules, such as the pressurized gas source 101 and the switching means 102, so that ventilation forms according to the prior art can be performed.

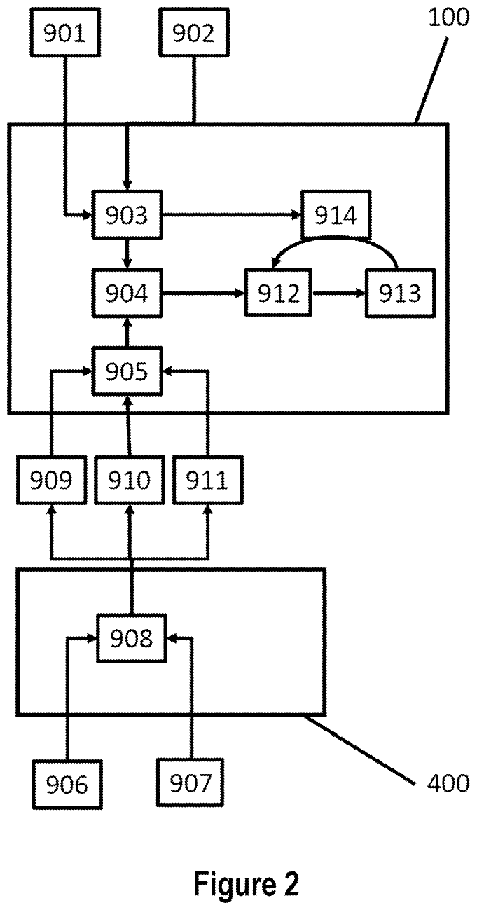

FIG. 2 shows a schematic illustration of the consideration of individual data streams with respect to the therapy. A therapy device 100 is coupled at least for data exchange with a ventilator 400 by way of example.

User inputs 901, for example input via a user interface 107 on the therapy device 100, and sensor values 902, which are registered, for example, via one or more sensors 105 of the therapy device 100, are processed, for example, in an internal processing 903. The processed data can be provided, inter alia, to a therapy reminder and/or therapy start mechanism and/or a therapy repetition mechanism. The start mechanism or reminder mechanism can have a start/a reminder 904 of the therapy follow on the basis of the data situation of the internal processing 903, for example, which results in a performance 912 of the therapy. During the therapy, for example, further sensor values 902 are recorded, so that a repetition 914 can be provided via the therapy repetition mechanism after end 913 of the therapy. Alternatively or additionally, a repetition 914 of the therapy can also take place on the basis of the user inputs 901.

With a coupling of the therapy device 100 to a ventilator 400, data, values, and/or information of the ventilator 400 can also be taken into consideration for the therapy. For example, the ventilator 400 registers separate user inputs 906 and/or sensor values 907, which are prepared and/or evaluated, for example, in an internal processing 908 in the ventilator 400. In some embodiments, it can be provided that the ventilator 400 determines control commands 909 from the user inputs 906 and/or sensor values 907. The optionally prepared user inputs 911 and sensor values 910 and possible control commands 909 are transmitted to the coupled therapy device 100.

A processing 905 of external data, such as the data from the ventilator 400, is provided, for example, in the therapy device 100. It can be provided here that at least the user inputs 911 and/or the sensor values 910 are evaluated, for example, to effectuate a start/a reminder 904 of the therapy. For example, the user inputs 911 and/or the sensor values 910 of the ventilator 400 can indicate that a certain amount of secretion has collected and coughing up would be reasonable.

Moreover, the control commands 909 of the ventilator 400 to the therapy device 100 can be implemented by the processing 905. It can be provided, for example, that the internal processing 908 of the data in the ventilator 400 establishes in an evaluation that a therapy is to be performed. The processing 905 of the control commands 909 in the therapy device 100 can thus have the result that a start/a reminder 904 of the therapy is initiated.

Alternatively or additionally, the therapy device 100 can also adapt the therapy settings and/or the duration between two therapys or until the next therapy on the basis of the user data 901, 911 and/or sensor data 902, 910.

In some embodiments, it can moreover be provided that therapy settings (duration, form, intensity, duration between two therapys) can be performed directly via user inputs 906 on the ventilator 400 and these can be transmitted to the therapy device 100, for example as control commands 909.

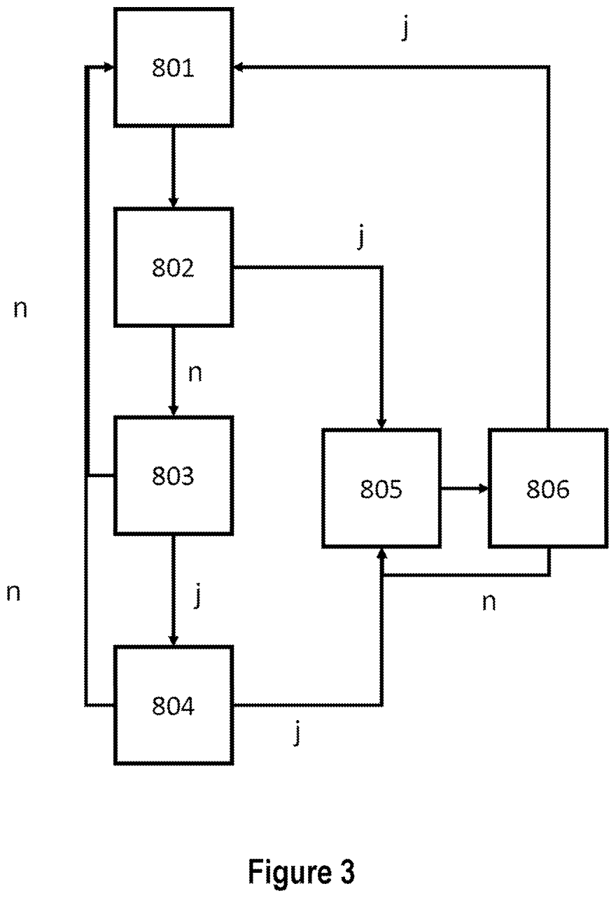

FIG. 3 schematically shows a very simplified decision tree for decision finding as to whether the therapy start, a therapy reminder, and/or a therapy repetition is to be activated. In FIG. 3, the letter j represents a positive response to the query, the letter n a negative response.

Proceeding from the starting point 801, for example, the first query 802 is a query about the duration passed since the last therapy, as to whether a certain threshold value is reached or exceeded. If the threshold value for a duration since the last therapy is reached, the query 802 is thus assessed with “j”. If the query 802 for a threshold value of the duration is positive, the therapy start and/or a therapy reminder 805 thus follows. Upon an assessment with “n”, the next query takes place.

The next query 803 is, for example, the question as to whether the therapy device 100 is coupled for data exchange with a ventilator 400 or corresponding data are transmitted from the ventilator 400. If no data coupling exists, a negative response “n” takes place and the decision finding begins again at the starting point 801. Upon a positive response, the next query follows.

For example, the following query 804 is a query as to whether the data transmitted from the ventilator 400 indicate an accumulation of secretion which should be coughed up. The evaluation of the data can take place, for example, in the ventilator 400 or by the therapy device 100. Upon a negative response, the decision finding begins again at the starting point 801. Alternatively or additionally, further queries can also be provided.

If the data situation indicates an accumulation of secretion, the query 804 is thus positively answered and a therapy reminder and/or a therapy start 805 follows.

During and/or after the therapy it is provided, for example, that a query 806 about the therapy result, for example whether the therapy has resulted in adequate coughs, follows. If the therapy has been adequate, the decision finding begins again for the start/the reminder of the therapy at the starting point 801. If the therapy should not have been adequate, a renewed therapy start and/or a therapy reminder 805 can be provided.

In addition to the sequences shown, the decision tree can be expanded arbitrarily. For example, further data, values, and/or information can be queried to make a decision. It can also be provided, for example, that the determination of the duration which is to pass until the next therapy is determined on the basis of these data/values/information and is introduced, for example, between beginning 801 and the first query 802 after the duration.

If, for example, a pneumatic coupling between therapy device 100 and ventilator 400 is also present, further corresponding queries can be incorporated, for example as to whether a change of the patient interface is necessary.

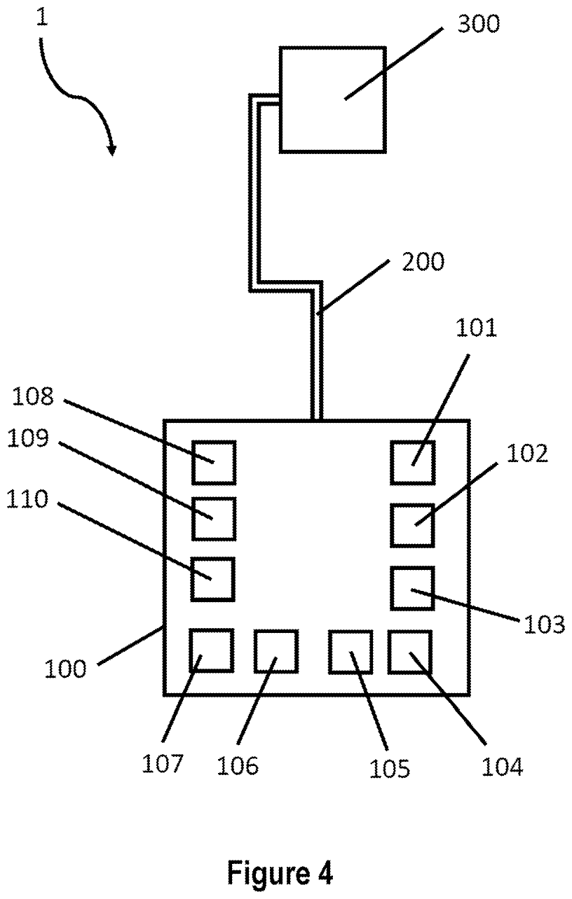

FIG. 4 shows an exemplary system 1 having a therapy device 100, wherein the therapy device 100 having the controllable pressurized gas source 101 is configured to perform at least one insufflation of the patient 300 via a hose system 200. The therapy device 100 is designed, for example, to perform a therapy. In some embodiments, the therapy device 100 is a combination device, which can at least temporarily also perform the ventilation of the patient 300 as a ventilator.

The system 1 furthermore comprises switching means 102, via which overpressure can be applied to the hose system 200, for example, to perform an insufflation. The system 1 with the switching means 102 is configured to suddenly stop the application of overpressure to the hose system 200 and to force an expiration and/or a cough of the patient 300.

The exemplary therapy device 100 furthermore comprises a memory 103 for storing data, values, and information which can be input, for example, via a user interface 107, a sensor 105, an evaluation unit 106, and/or via a ventilator (not shown) coupled for data exchange. The memory 103 is designed to provide data, values, and/or information to the control unit 104, the evaluation unit 106, the therapy reminder mechanism 108, the therapy repetition mechanism 109, and/or the therapy start mechanism 110. The therapy reminder mechanism 108, the therapy repetition mechanism 109, and/or the therapy start mechanism 110 can be provided, for example, as software modules on the evaluation unit 106 and/or the control unit 104. The therapy mechanisms 108, 109, 110 can in some embodiments also comprise separate hardware modules and/or can be installed on a common hardware module. It can also be provided that data, values, and/or information from the memory 103 can be displayed via the user interface 107. The data, values, and information stored in the memory 103 can contain, inter alia, at least parameters on the therapy, such as therapy length, therapy intensity, and/or therapy form. Data with respect to the patient 300 can also be comprised.

For example, the therapy device 100 comprises three therapy mechanisms: a therapy reminder mechanism 108, a therapy repetition mechanism 109, and a therapy start mechanism 110. The individual mechanisms were already described in detail above and will only be explained briefly and by way of example at this point.

The therapy reminder mechanism 108 is configured to remind the patient 300 and/or a caregiver of the therapy on the basis of a data situation. For example, at least one duration since the last performed therapy is used as the data situation. The therapy reminder mechanism 108 generates for this purpose, for example, a corresponding notification, for example for display on the user interface 107. The notification can notify, for example, that a certain duration has passed since the last therapy and accordingly a new therapy is to be started. The notification can also comprise a step-by-step instruction for this purpose, which displays the steps of the therapy, such as which preparations have to be made, etc. In some embodiments, it can be provided that the patient 300 or a caregiver can mark the reminder as seen and/or can postpone it, so that a new notification is generated after a specific duration.

In some embodiments, the therapy reminder mechanism 108 can also be configured to prepare the therapy device 100 for the therapy. For example, a therapy program can be loaded, therapy settings or therapy parameters can be automatically adjusted and/or the therapy device can be warmed up/started up. It can also be provided that the therapy reminder mechanism 108 prepares the therapy enough that the patient 300 and/or caregiver only has to confirm the performance of the therapy, for example via the user interface 107. As already explained, in addition to the duration, other factors, data, values, and/or information can also be incorporated in the data situation.

The therapy repetition mechanism 109 is configured, for example, to start a repetition and/or an extension of the therapy on the basis of a data situation. The data situation can comprise in particular values of the sensor 105 here, which are registered during the therapy. For example, the therapy repetition mechanism 109 can conclude by an evaluation of the data that the therapy is not adequate or is not yet adequate. If the therapy is not adequate, a repetition can be provided. For the repetition, for example, the same therapy settings or parameters can be reused. However, it can also be provided that certain parameters, such as intensity and/or therapy form, are changed to achieve a better therapy. If the therapy is not yet adequate, it can be provided, for example, that the therapy is extended by at least one cycle, it can also be provided that it is extended by at least one or more maneuvers. In some embodiments, a protective mechanism is provided to prevent an excessively long therapy, even if it is not yet adequate. In this case, for example, a notification or an alarm can additionally be generated that a maximum therapy length is reached, but it is not yet adequate.

The therapy start mechanism 110 is configured, for example, to automatically start the therapy. The therapy start mechanism 110 can be constructed similarly to the therapy reminder mechanism 108 with respect to the data situation. An automatic start of the therapy is possible in particular if the therapy device 100 is pneumatically coupled with a ventilator 400 (not shown). The therapy start mechanism 110 can then provide an automatic start, for example, when a maximum duration and/or maximum number of reminders of the therapy have passed.

To perform the therapy, the pressurized gas source 101 and the switching means 102 are activated in accordance with the specifications, which are stored, for example, in the memory 103. During the performance of the therapy, first an overpressure gas for insufflation is provided to the patient 300 via the hose system 200, so that the patient 300 can inhale and air can pass behind the secretion to be coughed up. The provision of the overpressure gas is then stopped suddenly by the therapy device 100 or by the switching means 102, so that a cough is triggered in the patient 300, by which the secretion is to be transported away. In the course of a therapy, multiple coughs or cycles are generally provided, wherein a cycle consists of an inhalation (inspiration) and an exhalation (expiration) or cough. Depending on the therapy settings, the intensity, such as the pressure difference and/or the switching speed, of the coughs can vary. A pause between 0.1 seconds and 10 seconds is generally provided between two therapy cycles. In some embodiments, a therapy can also be divided into multiple maneuvers, wherein a maneuver in turn comprises multiple cycles. It can also be provided that the pause is greater between two maneuvers them between two cycles, for example also greater than 10 seconds.

After ending the therapy, it can be provided that the patient 300 and/or a caregiver can input feedback on the therapy via the user interface 107, for example the subjective feeling of the effect of the therapy. A further performance of the therapy can also be requested.

In some embodiments, the system 1 or the therapy device 100 is designed to perform a ventilation of the patient. For example, an overpressure ventilation or a respiration assistance of the patient can be performed via the source for gas for generating an overpressure. In some embodiments, the therapy device 100 is thus a ventilator and therapy device combined. The control unit 104 is configured and designed here so that it activates the further modules, such as the pressurized gas source 101 and the switching means 102, so that ventilation forms according to the prior art can be performed.

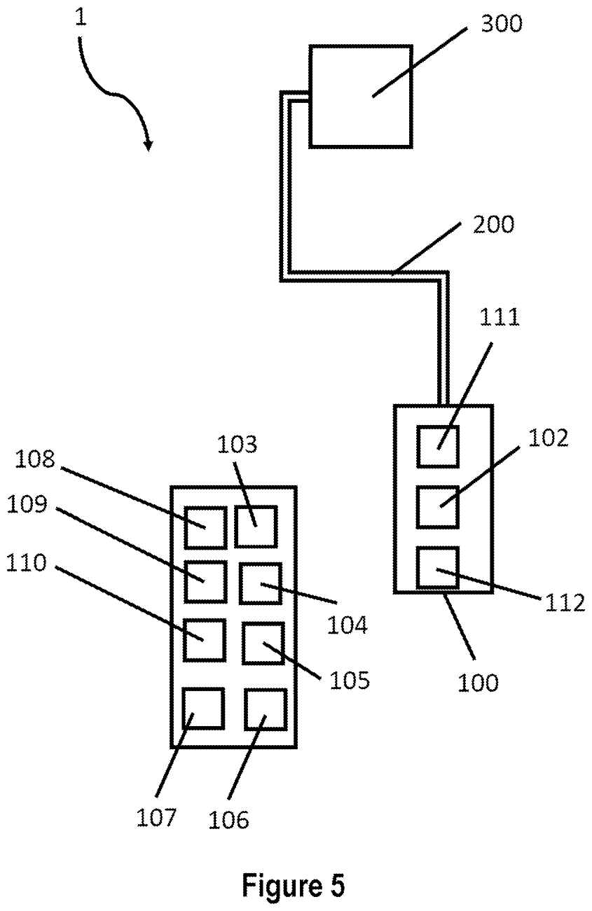

FIG. 5 shows an exemplary system 1 having a therapy device 100, wherein the therapy device 100 comprises a drain for gas for generating a negative pressure in the hose system 200. Via the negative pressure it is possible, for example, to extract secretion from the mouth and/or throat area of the patient 300.

In the system 1 shown by way of example, the control unit 104 having the mechanisms 108, 109, 110 as well as the memory 103, the sensor 105, the evaluation unit 106, and the user interface 107 is arranged at least spatially independent from the therapy device 100. In some embodiments, the control unit 104 having the mechanisms 108, 109, 110 as well as the memory 103, the sensor 105, the evaluation unit 106, and the user interface 107 can be arranged in a ventilator or combined as a separate device, which monitors the respiration and/or at least the secretion situation of the patient.

The exemplary system 100 comprises a memory 103 for storing data, values, and information, which can be input, for example, via a user interface 107, a sensor 105, an evaluation unit 106, and/or via a ventilator (not shown) coupled for data exchange. The memory 103 is designed to provide data, values, and/or information to the control unit 104, the evaluation unit 106, the therapy reminder mechanism 108, the therapy repetition mechanism 109, and/or the therapy start mechanism 110. The therapy reminder mechanism 108, the therapy repetition mechanism 109, and/or the therapy start mechanism 110 can be provided, for example, as software modules on the evaluation unit 106 and/or the control unit 104. The therapy mechanisms 108, 109, 110 can in some embodiments also comprise separate hardware modules and/or can be installed on a common hardware module. It can also be provided that data, values, and/or information from the memory 103 can be displayed via the user interface 107. The data, values, and information stored in the memory 103 can contain, inter alia, at least parameters on the therapy, such as therapy length, therapy intensity, and/or therapy form. Data with respect to the patient 300 can also be comprised.

For example, the system 100 comprises three therapy mechanisms: a therapy reminder mechanism 108, a therapy repetition mechanism 109, and a therapy start mechanism 110. The individual mechanisms were already described in detail above and will only be explained briefly and by way of example at this point.

The therapy reminder mechanism 108 is configured to remind the patient 300 and/or a caregiver of the therapy on the basis of a data situation. For example, at least one duration since the last performed therapy is used as the data situation. The therapy reminder mechanism 108 generates for this purpose, for example, a corresponding notification, for example for display on the user interface 107. The notification can notify, for example, that a certain duration has passed since the last therapy and accordingly a new therapy is to be started. The notification can also comprise a step-by-step instruction for this purpose, which displays the steps of the therapy, such as which preparations have to be made, etc. In some embodiments, it can be provided that the patient 300 or a caregiver can mark the reminder as seen and/or can postpone it, so that a new notification is generated after a specific duration.

In some embodiments, the therapy reminder mechanism 108 can also be configured to prepare the therapy device 100 for the therapy. For example, a therapy program can be loaded, therapy settings or therapy parameters can be automatically adjusted and/or the therapy device can be warmed up/started up. It can also be provided that the therapy reminder mechanism 108 prepares the therapy enough that the patient 300 and/or caregiver only has to confirm the performance of the therapy, for example via the user interface 107. As already explained, in addition to the duration, other factors, data, values, and/or information can also be incorporated in the data situation.

The therapy repetition mechanism 109 is configured, for example, to start a repetition and/or an extension of the therapy on the basis of a data situation. The data situation can comprise in particular values of the sensor 105 here, which are registered during the therapy. For example, the therapy repetition mechanism 109 can conclude by an evaluation of the data that the therapy is not adequate or is not yet adequate. If the therapy is not adequate, a repetition can be provided. For the repetition, for example, the same therapy settings or parameters can be reused. However, it can also be provided that certain parameters, such as intensity and/or therapy form, are changed to achieve a better therapy. If the therapy is not yet adequate, it can be provided, for example, that the therapy is extended by at least one cycle. In some embodiments, a protective mechanism is provided to prevent an excessively long therapy, even if it is not yet adequate. In this case, for example, a notification or an alarm can additionally be generated that a maximum therapy length is reached, but it is not yet adequate.

The therapy start mechanism 110 is configured, for example, to automatically start the therapy. The therapy start mechanism 110 can be constructed similarly to the therapy reminder mechanism 108 with respect to the data situation. An automatic start of the therapy is possible in particular if the therapy device 100 is pneumatically coupled with a ventilator or the therapy device 100 is integrated in a ventilator. The therapy start mechanism 110 can then provide an automatic start, for example, when a maximum duration and/or maximum number of reminders of the therapy have passed.

To perform the therapy, a negative pressure is generated at the hose system 200 by the drain 111, wherein the negative pressure can be provided to the hose system 200 by the switching means 102 and the provision can also be stopped. The hose system 200 is, for example, a hose, optionally with a special end piece for extracting secretion. The secretion is extracted from the patient via the hose and possibly collected in a collection container 112 in or on the therapy device 100.

After ending the therapy, it can be provided that the patient 300 and/or a caregiver can give feedback on the therapy via the user interface 107, for example the subjective feeling of the effect of the therapy. A further performance of the therapy can also be requested.

In some embodiments, the therapy device 100 in the system 1 can also be combined and/or coupled with a ventilator. For example, the ventilator can be configured to perform a ventilation of the patient via a tracheal cannula. The therapy device 100 can be combined with the ventilator and/or integrated in the ventilator here so that it is possible to extract secretion via, for example, the tracheal cannula.

### LIST OF REFERENCE SIGNS

1 system
100 therapy device
101 pressurized gas source
102 switching means
103 memory
104 control unit
105 sensor
106 evaluation unit
107 user interface
108 therapy reminder mechanism
109 therapy repetition mechanism
110 therapy start mechanism
111 drain
112 collection container
801 starting point
802 query about passed duration
803 query about data exchange
804 query about secretion accumulation
805 start/reminder of therapy
806 query about therapy result
901 user inputs
902 sensor values
903 internal processing
904 start/reminder mechanism
905 processing of external data
906 user inputs
907 sensor values
908 internal processing
909 control commands
910 sensor values
911 user inputs
912 performance of the therapy
913 end of the therapy
914 repetition mechanism

1.-18. (canceled)

19. A therapy system, wherein the system comprises at least one therapy device for performing a therapy and at least one hose system for a gas-conducting connection between the therapy device and a patient, wherein the therapy device comprises at least one source for gas for generating an overpressure and/or at least one drain for gas for generating a negative pressure and at least temporarily provides an overpressure and/or negative pressure to the hose system to perform the therapy, and wherein the system furthermore comprises at least one control unit, at least one user interface, at least one memory, and at least one sensor, the control unit comprising at least one therapy mechanism selected from a therapy start mechanism for starting a therapy, a therapy reminder mechanism for reminding of the therapy, and a therapy repetition mechanism for extending and/or repeating the therapy, each of the therapy mechanisms being based on user actions, patient data, and/or therapy data.

20. The system of claim 19, wherein the therapy comprises one or more of a cough therapy, a cough assistance, an insufflation, an insufflation with following exsufflation, and an extraction of secretion from a mouth or airways.

21. The system of claim 19, wherein the control unit is configured to automatically adapt a frequency of the reminder of the therapy and/or the start of the therapy via the user interface and/or via an additional interface and/or from data characterizing a patient from the memory.

22. The system of claim 19, wherein based on determined data, the therapy is automatically started, the start of the therapy is offered to a patient or a reminder of the therapy is given if the determined data indicate that a secretion removal from airways by a therapy would be expedient.

23. The system of claim 22, wherein the determined data comprise one or more of measured values and stored values for SpO2, pulse, CO2, and a changing pressure and/or volume flow behavior.

24. The system of claim 19, wherein the therapy device is coupled with a ventilator or the therapy device and a ventilator are combined as one device and data for the at least one therapy mechanism are provided by the ventilator.

25. The system of claim 24, wherein a determined effect of performed therapies on a profile of at least one of the variables SpO2, CO2, flow, tidal volume, signal-to-noise ratio (SNR) changes ventilation parameters on the ventilator.

26. The system of claim 24, wherein the therapy device and the ventilator are pneumatically coupled, so that a ventilation and a machine insufflation and exsufflation can be directly combined.

27. The system of claim 26, wherein the reminder of the therapy or the start of the therapy takes place exclusively or additionally via the coupled ventilator.

28. The system of claim 19, wherein data on which the at least one therapy mechanism is based comprise an SpO2 value, at least one of the following functions being executed when the SpO2 value reaches an SpO2 threshold value: reminder of the therapy, start of the therapy, repetition of the therapy, extension of the therapy.

29. The system of claim 19, wherein an effect of a performed therapy on a profile of at least one of the following variables is stored and/or processed: SpO2, CO2, pressure, flow, tidal volume, acoustic signals, signal-to-noise ratio (SNR) in the therapy device and/or in a coupled ventilator, the storage and/or processed data being taken into consideration in decisions on at least one of the following functions: start of the therapy, repetition of the therapy, reminder of the therapy.

30. The system of claim 19, wherein the system is configured to evaluate a signal-to-noise ratio (SNR) of pressure and/or flow signals and to initiate or extend a start of the therapy or a reminder of the therapy if the SNR value reaches an SNR threshold value.

31. The system of claim 19, wherein, based on determined data or based on data from past cough maneuvers, repetitions of insufflations and exsufflations are performed automatically and/or offered to a patient if these data indicate that further secretion should be removed from the airways.

32. The system of claim 31, wherein determined data comprise one or more of measured values and stored values on one or more of pressure, flow, tidal volume, SpO2, pulse, CO2, and/or data from past maneuvers comprise a cough flow.

33. The system of claim 19, wherein determined and/or stored data are uploaded in a cloud-based system, cloud-based algorithms make therapy-related decisions, and these decisions are in turn provided to the therapy device from the cloud for adaptation of the therapy or of starting points in time of the therapy.

34. The system of claim 19, wherein a user-side feedback on a subjective perception of automatically performed starting procedures of the therapy and/or repetitions of the therapy and/or reminders of the therapy at the user interface and/or via an app on a mobile terminal coupled with the therapy device is possible, the user-side feedback being stored and/or processed and being taken into consideration in following decisions on the start of the therapy and/or the repetition of the therapy and/or the reminder of starting the therapy.

35. The system of claim 19, wherein a cough therapy as a result of the reminder of the therapy can be started and/or repeated and/or ended by the therapy device on a user side via a remote control and/or an app on a mobile terminal coupled with the therapy device.

36. The system of claim 19, wherein an automatic start of the therapy is provided on the basis of determined data, therapy parameters being automatically adapted on the basis of the determined data.

37. The system of claim 19, wherein the therapy device is configured to at least temporarily provide overpressure for an insufflation and/or at least temporarily provide negative pressure for extracting secretion and/or for an exsufflation to the hose system.

38. The system of claim 19, wherein the at least one control unit, the at least one memory, the at least one user interface, and the at least one sensor are a part of the therapy device.
